(19) United States
(12) Patent Application Publication
(10) Pub. No.: US 2025/0312226 A1
(43) Pub. Date: Oct. 9, 2025

(54) CERVICAL TRACTION DEVICE

(71) Applicant: Brace Direct, LLC, Hixson, TN (US)

(72) Inventor: Lennie Alan Furr, Hixson, TN (US)

(21) Appl. No.: 18/629,811

(22) Filed: Apr. 8, 2024

(51) Int. Cl. A61H 1/02; A61F 5/042

(52) U.S. Cl. CPC A61H 1/0296; A61F 5/042; A61H 2201/0134; A61H 2201/0192; A61H 2201/0221; A61H 2201/1611; A61H 2201/169

(57) ABSTRACT

A cervical traction device incorporating temperature regulation during a physical therapy session includes a traction base body, a resting assembly, and a first and second pad for temperature regulation removably received in a first and second removable cushion pocket, respectively. The first and second pads are configured to maintain respective temperatures during the session.

### BACKGROUND OF THE INVENTION

In the modern world, a growing number of people lead sedentary lifestyles and spend extended periods using digital devices. Consequently, discomfort and pain in the neck, spine, or back have become quite common. Additionally, psychological factors and stress levels can worsen neck pain symptoms, further contributing to the prevalence of this condition.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification and serve to illustrate further embodiments of concepts that include the claimed invention and explain various principles and advantages of those embodiments.

FIG. 1 is a perspective view of a cervical traction device in accordance with some embodiments.

FIG. 2 is a perspective view of a traction base body of a cervical traction device in accordance with some embodiments.

FIG. 3 is a perspective view of a resting assembly of a cervical traction device in accordance with some embodiments.

FIG. 4 is a top view of a cervical traction device in accordance with some embodiments.

FIG. 5 is a side view of a cervical traction device in accordance with some embodiments.

FIG. 6 is an exploded view of a cervical traction device in accordance with some embodiments.

FIG. 7 is a flowchart of a method for temperature regulation of a cervical traction device in accordance with some embodiments.

Skilled artisans will appreciate that elements in the figures are illustrated for simplicity and clarity and have not necessarily been drawn to scale. For example, the dimensions of some of the elements in the figures may be exaggerated relative to other elements to help improve understanding of embodiments of the present invention.

The device and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

The embodiments described herein are related to a cervical traction device and, in particular, to a cervical traction device that regulates temperature during physical therapy sessions.

In one aspect, a cervical traction device incorporating temperature regulation during a physical therapy session is described. The cervical traction device includes a traction base body, a resting assembly, a first pad for temperature regulation, and a second pad for temperature regulation. The traction base body further includes a shoulder support unit with a first fastener and a first removable cushion pocket with a first opening. The traction base body also includes a slider, a pneumatic cylinder coupled to the slider, and an angled block installed at one end of the traction base body to generate a desirable angle.

The resting assembly mechanically connects to the slider of the traction base body and further includes a headrest unit including a second fastener, a second removable cushion pocket with a second opening, and a neck support unit positioned in between the headrest unit and the shoulder support unit which is configured to be located opposite to the angled block of the traction base body.

The first pad for temperature regulation is positioned within the first opening of the first removable cushion pocket and the second pad for temperature regulation is positioned within the second opening of the second removable cushion pocket. In addition, the device includes means for controlling the pneumatic cylinder to actuate the slider to cause coordinated movement of the neck support unit and the headrest unit.

In another aspect, a method for temperature regulation of a cervical traction device during a physical therapy session is described. The method includes securing the first removable cushion pocket on the shoulder support unit using the first fastener and securing the second removable cushion pocket on the headrest unit with the second fastener.

The method further includes inserting the first pad for temperature regulation inside the first removable cushion pocket via the first opening and inserting the second pad for temperature regulation inside the second removable cushion pocket via the second opening. The method, moreover, includes fastening the first and second openings of the first and second removable cushion pockets, followed by retaining the first and second pads inside the first and second removable cushion pockets during the physical therapy session. During temperature regulation of the cervical traction device, the first and second pads are configured to maintain a similar or variable temperature.

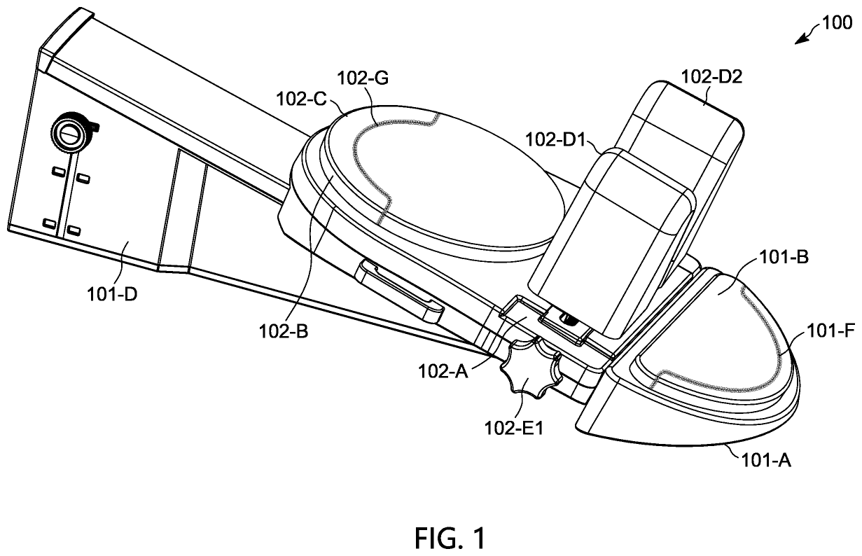

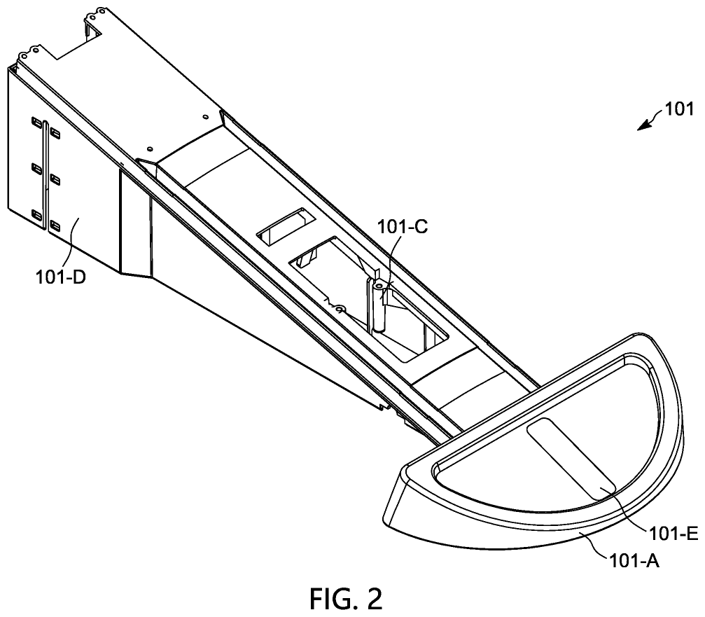

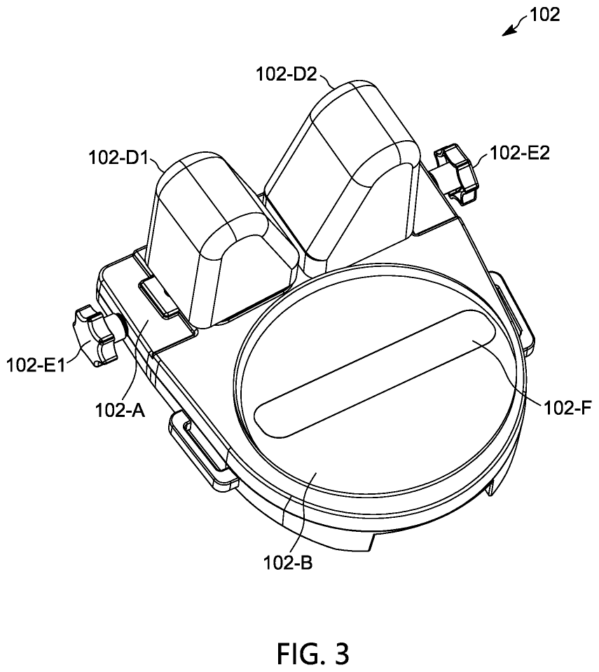

Referring to FIG. 1 to FIG. 3, a cervical traction device 100 is illustrated and described. The cervical traction device 100 includes a traction base body 101 (further described in FIG. 2) and a resting assembly 102 (further described in FIG. 3). The traction base body 101 further includes a shoulder support unit 101-A and a first removable cushion pocket 101-B. The traction base body 101 also includes a slider 101-C, a pneumatic cylinder (not illustrated) coupled to the slider 101-C, and an angled block 101-D installed at one end of the traction base body 101 to generate a desirable angle.

The resting assembly 102 mechanically connects to the slider 101-C of the traction base body 101 and further includes a headrest unit 102-B having a second removable cushion pocket 102-C, and a neck support unit 102-A. The neck support unit 102-A is positioned between the headrest unit 102-B and the shoulder support unit 101-A, configured to be opposite the angled block 101-D of the traction base body 101.

The neck support unit 102-A further includes a pair of support blocks 102-D1 and 102-D2 to receive a neck portion of a user during a physical therapy session. The neck support unit 102-A also includes a pair of adjustable knobs 102-E1 and 102-E2 mechanically connected to the pair of support blocks 102-D1 and 102-D2 using a plurality of connectors (not illustrated). For example, the adjustable knobs 102-E1 mechanically connect to the support block 102-D1 and the adjustable knobs 102-E2 mechanically connect to the support block 102-D2 using the plurality of connectors.

The plurality of connectors may comprise one or more selected from a group comprising a threaded connector, a quick-release pin connector, a clamp and latch connector, and a swivel joint and a ball joint connector. It will be appreciated that the plurality of connectors can include any connectors now known or in the future developed. The plurality of connectors can operate using a mechanism including a rack and pinion mechanism, a threaded rods and nuts mechanism, and an adjustable clamp or locking mechanism. It will be appreciated that the mechanism can include any mechanism now known or in the future developed.

The cervical traction device 100 further includes means for controlling the pneumatic cylinder (not illustrated) to actuate the slider 101-C to cause coordinated movement of the neck support unit 102-A and the headrest unit 102-B, where the means for controlling the pneumatic cylinder comprises one or more selected from a group comprising an air pipe and a crank device. It will be appreciated that the means for controlling the pneumatic cylinder can include any means now known or in the future developed.

Referring to FIG. 2 in light of FIG. 1, a perspective view of the traction base body 101 of the cervical traction device 100 is described. The shoulder support unit 101-A removably receives the first removable cushion pocket 101-B on the shoulder support unit 101-A using a first fastener 101-E. The first fastener 101-E comprises one or more selected from a group comprising a hook-and-loop fastener, a snap closure, a button closure, and an elastic element. The first removable cushion pocket, 101-B, is constructed from a single or a multilayer water-resistant product material selected from a group of a cotton, a polyester, a copper fabric, a quilting fabric, a silk, a polyethylene, and a polymer. It will be appreciated that the first fastener 101-E can include any fasteners now known or in the future developed.

Referring to FIG. 3 in light of FIG. 1, a perspective view of the resting assembly 102 of the cervical traction device 101 is described. The headrest unit 102-B removably receives the second removable cushion pocket 102-C using a second fastener 102-F. The second fastener, 102-F, comprises one or more selected from a group comprising a hook-and-loop fastener, a snap closure, a button closure, and an elastic element. The second removable cushion pocket 102-C is constructed from a single or a multilayer water-resistant product material selected from a group of a cotton, a polyester, a copper fabric, a quilting fabric, a silk, a polyethylene, and a polymer. It will be appreciated that the second fastener 102-F can include any fasteners now known or in the future developed.

The pair of adjustable knobs 102-E1 and 102-E2 can also be a pusher knob without limiting to any shape or size. The pair of adjustable knobs 102-E1 and 102-E2 are configured to allow the user to adjust a space in between the pair of support blocks 102-D1 and 102-D2, thereby allowing the neck portion of the user to rest in between the pair of support blocks 102-D1 and 102-D2. The pair of adjustable knobs 102-E1 and 102-E2 are configured to control a traction force applied to the neck portion of the user, wherein the user can rotate or push the pair of adjustable knobs 102-E1 and 102-E2 to the user's neck shape and size.

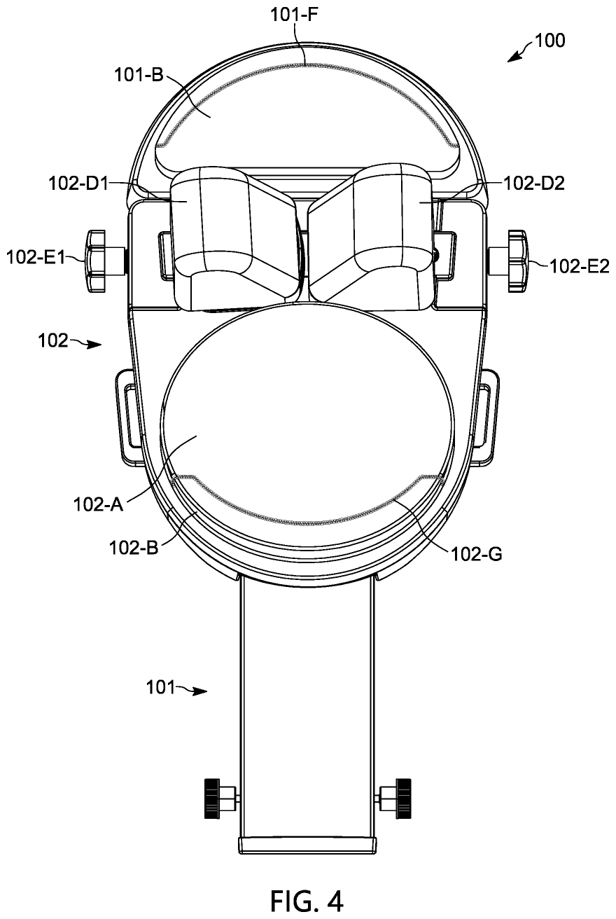

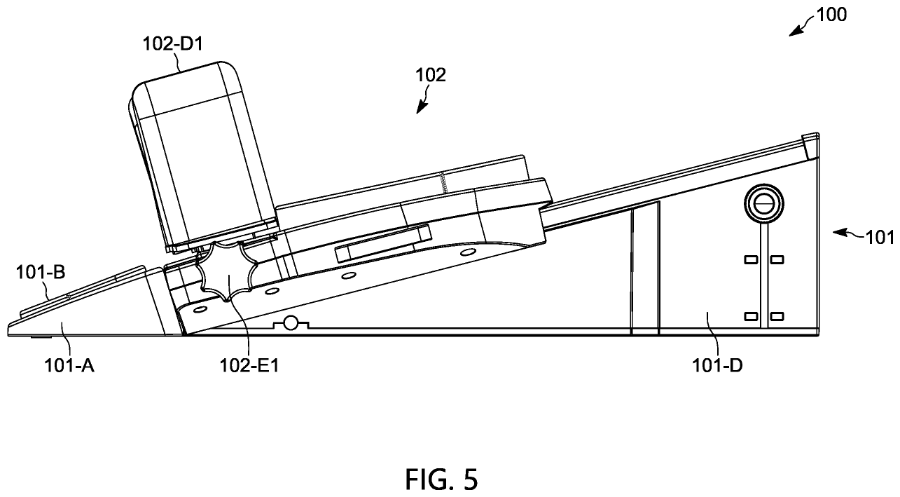

Referring to FIGS. 4 and 5, the neck support unit 102-A is positioned between the headrest unit 102-B and the shoulder support unit 101-A, configured to be opposite the angled block 101-D of the traction base body 101. The angled block 101-D facilitates targeted stretching, alignment correction, and muscle relaxation during the physical therapy session. The angled block 101-D further includes a plurality of knobs (not illustrated) mechanically connected to the traction base body 101 to adjust the angle of the traction base body 101, thereby allowing the user to rest the neck portion on the neck support unit 102-A, a head portion on the headrest unit 102-B, and a shoulder portion on the shoulder support unit 101-A. The plurality of knobs may connect to the traction base body 101 using a rotational mechanism, a linkage system, and a locking mechanism. It will be appreciated that the plurality of knobs can include any connection mechanisms now known or in the future developed.

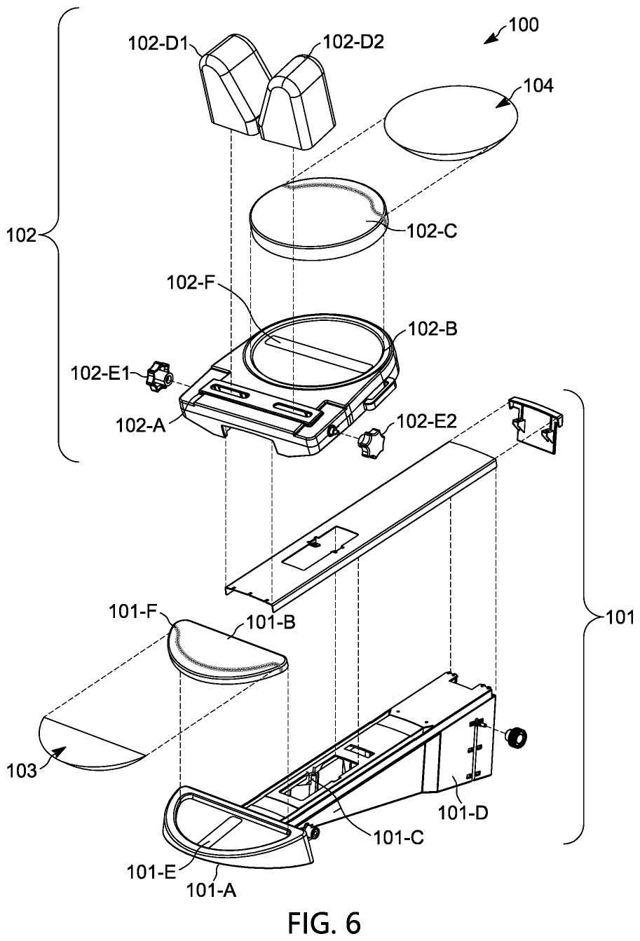

Referring to FIG. 6 in light of FIG. 1, an exploded view of a cervical traction device is described. The first removable cushion pocket 101-B includes a first opening 101-F to removably receive a first pad for temperature regulation 103. The first opening 101-F comprises one or more selected from a group comprising a circular opening, a semi-circular opening, and a snap pouch opening. It will be appreciated that the first opening 101-F can include any opening now known or in the future developed. The first pad for temperature regulation 103 is configured to maintain a first temperature during the physical therapy session, wherein the first temperature can be any temperature including a hot temperature or a cold temperature. The first pad for temperature regulation 103 comprises one or more selected from a group comprising an electric heating pad, an electric cooling pad, a heating gel pad, and a cooling gel pad. It will be appreciated that the first pad for temperature regulation 103 can include any pad now known or in the future developed.

The second removable cushion pocket 102-C includes a second opening 102-G to removably receive a second pad for temperature regulation 104. The second opening, 102-G (illustrated in FIG. 1), comprises one or more selected from a group comprising a circular opening, a semi-circular opening, and a snap pouch opening. It will be appreciated that the second opening 102-G can include any opening now known or in the future developed. The second pad for temperature regulation 104 is configured to maintain a second temperature during the physical therapy session, wherein the second temperature can be any temperature including a hot temperature or a cold temperature. It will be appreciated that the second temperature and the first temperature are independent of each other. For example, the first temperature can be a relatively hot temperature and the second temperature can be a relatively cooler temperature. The second pad for temperature regulation 104 comprises one or more selected from a group comprising an electric heating pad, an electric cooling pad, a heating gel pad, and a cooling gel pad. It will be appreciated that the second pad for temperature regulation 104 can include any pad now known or in the future developed.

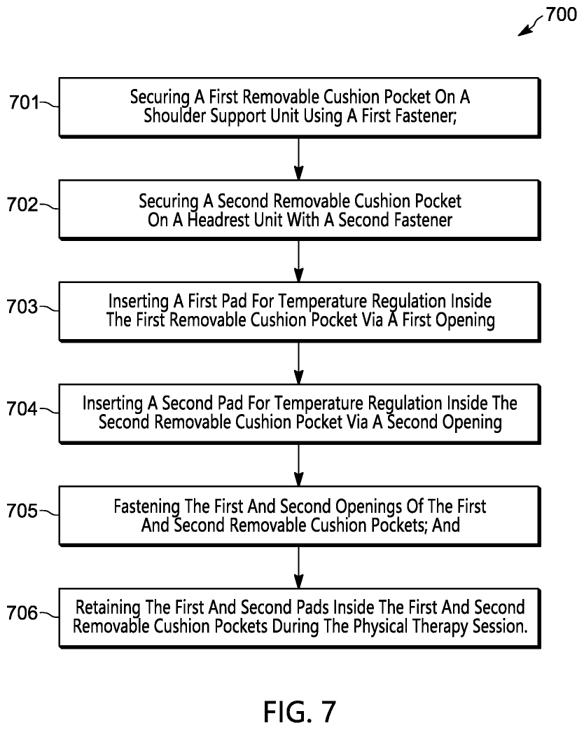

FIG. 7 is a flowchart of a method for temperature regulation of the cervical traction device 100 in accordance with some embodiments. The method for temperature regulation of the cervical traction device 100 will be described hereinafter with reference to FIG. 7 indicated as 700, beginning with operation 701. Operation 701 includes securing the first removable cushion pocket 101-B on the shoulder support unit 101-A using the first fastener 101-E. In operation 702, securing the second removable cushion pocket 102-C on the headrest unit 102-B with the second fastener 102-F. In operation 703, the method includes inserting the first pad for temperature regulation 103 inside the first removable cushion pocket 101-B via the first opening 101-F. Operation 704, includes inserting the second pad for temperature regulation 104 inside the second removable cushion pocket 102-C via the second opening 102-G. In operations 705 and 706, fastening the first 101-F and second 102-G openings of the first 101-B and second removable cushion pockets 102-C to retain the first 103 and second 104 temperature regulation pads inside during the physical therapy session.

According to some embodiments, the first pad for temperature regulation 103 and the second pad for temperature regulation 104 can be formed of any shape or size. In an alternate embodiment, the first pad for temperature regulation 103 and the second pad for temperature regulation 104 can be composed of additional or fewer components. In one of the embodiments, the first pad for temperature regulation 103 and the second pad for temperature regulation 104 are designed to be removably received by the first and second removable cushion pockets 101-B, 102-C of any size or shape.

According to an embodiment, the first pad for temperature regulation 103 and the second pad for temperature regulation 104 can be set to maintain a similar or variable temperature during the physical therapy session. The first pad for temperature regulation 103 and the second pad for temperature regulation 104 can be pre-set to the first and second temperature before inserting them in the first and second removable cushion pockets 101-B, 102-C using an external temperature regulation medium such as a microwave, and refrigeration devices. The first and second temperatures can be a hot temperature or a cold temperature, wherein the hot temperature increases blood flow, relaxes muscles, and soothes discomfort during the session, whereas the cold temperature numbs pain, decreases inflammation, and constricts blood vessels, thereby reducing swelling and tissue damage.

According to another embodiment, the user rests the shoulder portion on the first pad for temperature regulation 103, and the head portion on the second pad for temperature regulation 104, thereby receiving the first and second temperature to the respective portions during the physical therapy session.

In another embodiment, the cervical traction device 100 is used to alleviate cervical spine discomfort and associated symptoms in both clinical settings and home environments. In another embodiment, the physical therapy session can be performed in both clinical settings and home environments.

According to another embodiment, the cervical traction device 100 may include a head strap to secure the device 100 around the head portion and the neck portion of the user.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as critical, required, or essential features or elements of any or all the claims. The invention is defined solely by the appended claims, including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or device that comprises, has, includes, contains a list of elements does not include only those elements but may include other elements not expressly listed or inherent to such process, method, article, or device. An element preceded by “comprises . . . a”, “has . . . a”, “includes . . . a”, “contains . . . a” does not, without more constraints, preclude the existence of additional identical elements in the process, method, article, or device that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

We claim:

1. A cervical traction device incorporating temperature regulation during a physical therapy session, the cervical traction device comprising:
a traction base body including:
a shoulder support unit including:
a first fastener, and
a first removable cushion pocket with a first opening;
a slider,
a pneumatic cylinder coupled to the slider, and
an angled block installed at one end of the traction base body to generate a desirable angle;
a resting assembly mechanically connected to the slider of the traction base body comprising:
a headrest unit including:
a second fastener, and
a second removable cushion pocket with a second opening, and
a neck support unit positioned in between the headrest unit and the shoulder support unit which is configured to be located opposite to the angled block of the traction base body;
a first pad for temperature regulation positioned within the first opening of the first removable cushion pocket;
a second pad for temperature regulation positioned within the second opening of the second removable cushion pocket; and
means for controlling the pneumatic cylinder to actuate the slider to cause coordinated movement of the neck support unit and the headrest unit.

2. The cervical traction device according to claim 1, wherein the neck support unit further includes:
a pair of support blocks to receive a neck portion of a user during a physical therapy session; and
a pair of adjustable knobs mechanically connected to the pair of support blocks.

3. The cervical traction device according to claim 1, wherein the first fastener comprises one or more selected from a group comprising a hook-and-loop fastener, a snap closure, a button closure, and an elastic element.

4. The cervical traction device according to claim 1, wherein the second fastener, wherein the fastener comprises one or more selected from a group comprising a hook-and-loop fastener, a snap closure, a button closure, and an elastic element.

5. The cervical traction device according to claim 1, the first and second pads for temperature regulation comprises one or more selected from a group comprising an electric heating pad, an electric cooling pad, a heating gel pad, and a cooling gel pad.

6. The cervical traction device according to claim 1, wherein the first pad for temperature regulation is configured to maintain a first temperature, wherein the first temperature can be a hot temperature or a cold temperature.

7. The cervical traction device according to claim 1, wherein the second pad for temperature regulation is configured to maintain a second temperature, wherein the second temperature can be a hot temperature or a cold temperature.

8. The cervical traction device according to claim 1, wherein the first and second removable cushion pockets are constructed from a single or a multilayer water-resistant product material selected from a group of a cotton, a polyester, a copper fabric, a quilting fabric, a silk, a polyethylene, and a polymer.

9. The cervical traction device according to claim 1, wherein the means for controlling the pneumatic cylinder comprises one or more selected from a group comprising an air pipe and a crank device.

10. A method for temperature regulation of a cervical traction device during a physical therapy session, the method comprising:
securing a first removable cushion pocket on a shoulder support unit using a first fastener;
securing a second removable cushion pocket on a headrest unit with a second fastener;
inserting a first pad for temperature regulation inside the first removable cushion pocket via a first opening;
inserting a second pad for temperature regulation inside the second removable cushion pocket via a second opening;
fastening the first and second openings of the first and second removable cushion pockets; and
retaining the first and second pads inside the first and second removable cushion pockets during the physical therapy session.
wherein the first and second pads for temperature regulation are configured to maintain a similar or variable temperature.

11. The method of claim 10, wherein the cervical traction device further includes a neck support unit positioned between the headrest unit and the shoulder support unit, thereby allowing a user to regulate temperature at a head and a neck portion.

12. The method of claim 10, wherein the method further comprising:
receiving the neck portion of the user via a pair of support blocks of the neck support unit; and
adjusting the neck portion of the user using a pair of adjustable knobs mechanically connected to the pair of support blocks.

13. The method of claim 10, wherein the first pad for temperature regulation is configured to maintain a first temperature, wherein the first temperature can be a hot temperature or a cold temperature.

14. The method of claim 10, wherein the second pad for temperature regulation is configured to maintain a second temperature, wherein the second temperature can be a hot temperature or a cold temperature.

15. The method of claim 10, wherein the first and second fasteners comprise one or more selected from a group comprising a hook-and-loop fastener, a snap closure, a button closure, and an elastic element.

16. The method of claim 10, wherein the first and second pads for temperature regulation comprise one or more selected from a group comprising an electric heating pad, an electric cooling pad, a heating gel pad, and a cooling gel pad.

17. The method of claim 10, wherein the first and second removable cushion pockets are constructed from a single or a multilayer water-resistant product material selected from a group of a cotton, a polyester, a copper fabric, a quilting fabric, a silk, a polyethylene, and a polymer.
